(19) United States
(12) Patent Application Publication
(10) Pub. No.: US 2022/0338538 A1
(43) Pub. Date: Oct. 27, 2022

(54) Portable Sealed Evaporator

(71) Applicant: Joseph Schlaegel, Sacramento, CA (US)

(72) Inventor: Joseph Schlaegel, Sacramento, CA (US)

(21) Appl. No.: 17/241,033

(22) Filed: Apr. 26, 2021

(51) Int. Cl. A24F 40/46; A24F 40/20; A24F 40/48; A24F 40/90; A24F 40/60; H05B 3/42

(52) U.S. Cl. CPC A24F 40/46; A24F 40/20; A24F 40/48; A24F 40/90; A24F 40/60; H05B 3/42; A61M 15/06

(57) ABSTRACT

A handheld evaporation apparatus having an evaporating heating element and a housing that encases the evaporating heating element. The evaporating heating element is adapted to achieve a temperature ranging from 250 to 2000 degrees Fahrenheit by heating and differential material composition. The housing includes a first end having an opening that allows heated air provided by the evaporating heating element to exit out through the first end. The housing further includes one or more air ducts on an outer surface of the housing, where the one or more air ducts allow ambient air outside the housing to enter into the housing and flow past the evaporating heating element so as to be heated by the evaporating heating element. In one example, the housing's first end has a semi-ovoid shape that presses against and seals a hemispherical-shaped case. In another example, the housing's first end includes an elastic band that surrounds the opening and presses against the inner surface of the bowl to create a substantially airtight seal.

### FIELD OF THE INVENTION

Multiple features relate to biomedical inhalation devices, and more specifically, to devices for extracting ingredients from a substance, such as a plant-based substance, through evaporation.

### BACKGROUND OF THE INVENTION

Extraction of natural ingredients from substances such as plant-based substances (e.g., herbs, botanicals, and cannabidiol (CBD) containing substances, etc.) has been shown to provide significant medical and health benefits. The benefits of directly inhaling the active ingredients into the circulatory system have desirable effects because such inhalation methods bypass the inefficiency of first digesting the product and process it into the bloodstream.

One common way of releasing the ingredients from a substance through inhalation is simply obtained from smoking the substance by applying fire directly to it to burn the substance and inhale the desired ingredients as they are released. However, several disadvantages arise in light of inhaling undesirable particulate along with the desired ingredients. More specifically, smoke particles enter and irritate the user's throat and lungs and introduce additional hazards to the user's body. Prolonged smoking introduces significant health risks in the mouth, throat, lungs and heart of the user. In addition, applying fire directly to the substance burns up a significant amount of the quantity resulting in using more than necessary to acquire the desired dosage and or effect.

Several devices which have reached the commercial marketplace seek to avoid the concerns caused by smoking an herbal product such as tobacco and/or marijuana. These devices seek to overcome the drawbacks of smoking by removing the smoke and carcinogens from the inhalation process as well as the odor caused by the smoke by vaporizing the herbal product instead of burning it with flame applied directly to the product. Some of these commercially available devices are large, unwieldy evaporation devices that are strictly devoted for evaporation.

One such example includes a vapor collecting chamber surrounding or coupled to a reservoir inside which the desired quantity of a herb oil is placed. A heating element called atomizer placed also inside the chamber is heated to vaporize the herb oil until the chamber is filled with a desired quantity of vapor. The vapors collected in the chamber may be withdrawn by inhaling from a tube connected to a passage leading to the interior of the chamber.

What is needed is a herbal evaporator that is portable, lightweight, effective, fast, and may be used safely without leakage.

### SUMMARY OF THE INVENTION

One feature provides a evaporation apparatus comprising an evaporating heating element and a housing encasing the evaporating heating element. The housing includes a first end having an opening adapted to allow heated air provided by the evaporating heating element to exit out through the first end. The housing further includes one or more air ducts on an outer surface of the housing, where the one or more air ducts are adapted to allow ambient air outside the housing to enter into the housing and flow past the evaporating heating element so as to be heated by the evaporating heating element. According to one aspect, the first end has a semi-ovoid shape. According to another aspect, the semi-ovoid shaped first end is adapted to press against and substantially seal a substantially hemispherical-shaped housing case.

According to one aspect, the first end includes a self-sealing elastic band that surrounds the opening and is adapted to press against and substantially seal a substantially hemispherical-shaped housing case. According to another aspect, the evaporating heating element is positioned in between the one or more air ducts and the first end. According to yet another aspect, the housing includes a second end opposite the first end, the second end including a Universal Serial Bus (USB) connector adapted to recharge a battery within the housing that provides power to the evaporating heating element.

According to one aspect, the second end includes a USB cover adapted to couple to the second end of the housing and cover the USB connector. According to another aspect, the evaporating heating element is positioned between two (2) and twenty (20) millimeters away from the first end within the housing. According to yet another aspect, the evaporating heating element is adapted to achieve a temperature ranging from 250 to 2,000 degrees Fahrenheit by various configurations of the design, which may include heating and differential materials compositions.

According to one aspect, the evaporating heating element is adapted to achieve a temperature ranging from 330 to 500 degrees Fahrenheit. According to another aspect, the housing is substantially cylindrical-shaped. According to yet another aspect, the evaporation apparatus further comprises a battery within the housing that supplies power to the evaporating heating element, and may include a button coupled to the housing, when present the button is adapted to activate the battery causing it to supply power to the evaporating heating element.

Another feature provides an apparatus comprising an evaporating heating element adapted to heat air, a housing encasing the heating element, the housing including a first end having an opening and at least one air flow hole, the heating element positioned between the opening and the air flow hole, and wherein a low air pressure region at the first end relative to ambient air outside of the air flow hole causes the ambient air to be drawn into the housing through the air flow hole and flow past the evaporating heating element and out through the opening at the first end. According to one aspect, the first end has a semi-ovoid shape that is adapted to press against an inner surface of a bowl. According to another aspect, the first end includes an elastic ring that surrounds the opening and is adapted to press against the inner surface of the case.

Another feature provides an apparatus comprising means for heating, an input interface adapted to activate the means for heating, and a housing at least partially enclosing the means for heating, the housing having a first end with a substantially semi-ovoid shape, the first end having an opening adapted to allow heat from the means for heating to radiate out from the first end. According to one aspect, the first end includes an elastic ring that surrounds the opening and is adapted to press against an inner surface of a bowl to create a substantially airtight seal. According to another aspect, the apparatus further comprises a chamber within the housing, and at least one air duct located on an outer surface of the housing that allows air to flow into the chamber through the air duct, and wherein the means for heating is positioned such that air flowing into the chamber from the air duct flows through the means for heating to be heated before it flows out through the opening.

### BRIEF DESCRIPTION OF THE DRAWINGS

FIG. 1 illustrates a perspective view of a first exemplary handheld evaporation apparatus (“evaporator”), with its USB cover on.

FIG. 2 illustrates a flat, slanted, and side view of the evaporator housing.

FIG. 3 illustrates a flat, top, slanted and side view of suction CAP of the evaporator.

FIG. 4 illustrates a flat, cutaway, slanted and top view of the heating element or atomizer of the evaporator.

FIG. 5 illustrates a sided, slanted, cross-sectional and top view of the atomizer electrode of the evaporator.

FIG. 6 illustrates a bottom view and a slanted view of the USB cover or the tail cap of the evaporator.

FIG. 7 illustrates a close-up view of the evaporator's bottom cover.

### DETAILED DESCRIPTION OF THE DRAWINGS

In the following description, specific details are given to provide a thorough understanding of the various aspects of the disclosure. However, it will be understood by one of ordinary skill in the art that the aspects may be practiced without these specific details. In other instances structures and techniques may not be shown in detail in order not to obscure the aspects of the disclosure.

The word “exemplary” is used herein to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation. The term “herbal product” is used herein to mean any plant-based substance or substance that includes plant material.

In operation a person desiring to vaporize an herbal product simply places the herbal product in any pipe, water pipe, or other device having a bowl configured to hold the herbal product in such a way as to expose at least a portion of the herbal product. The person then turns ON the evaporator, applies a first end of the evaporator closely to the exposed end of the bowl holding the herbal product, and inhales on the pipe. This causes ambient air to enter the evaporator through its air ducts and come out the first end having been heated to such a degree as to vaporize the herbal product. The vapors from the herbal product enter the pipe and may be consumed/inhaled by the person.

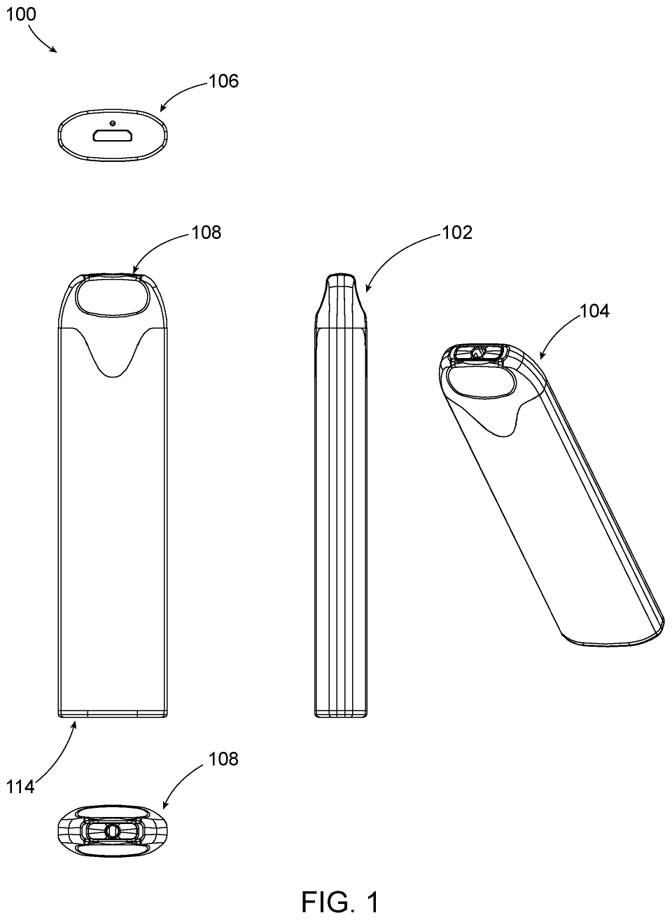

FIG. 1 illustrates various views of a handheld evaporation apparatus 100 (e.g., “evaporator”) according to one embodiment. More specifically, FIG. 1 illustrates a perspective view of the evaporator 100, and 102 and 104 illustrate side views of the evaporator 100. The evaporator 100 shown in FIG. 1 illustrates the evaporator 100 with its Universal Serial Bus (USB) cover 106 (e.g., “USB cap”) uncoupled/open. The top view 108 of the evaporator 100 is also shown in FIG. 1, illustrating the airflow opening.

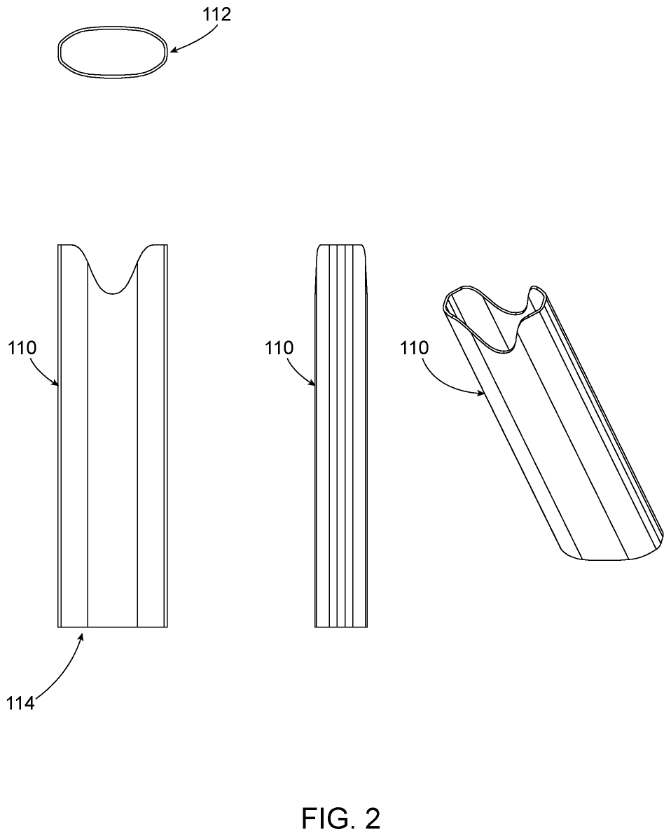

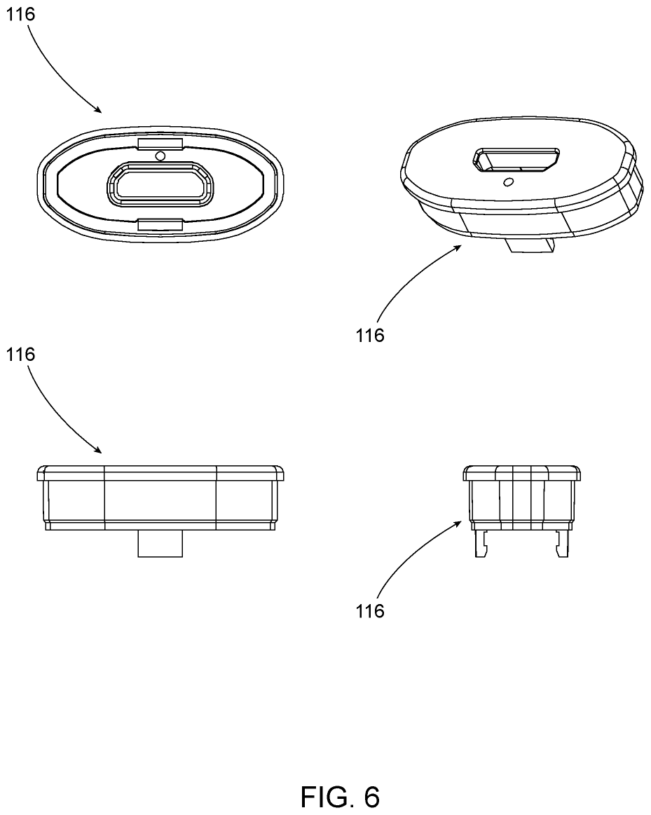

Referring to FIGS. 1, 2, 3, 4, and 6 the evaporator 100 may include, among other things, a housing 110 (e.g., also may be referred to as a “casing”), a first end 108, a second end 114 opposite the first end 108, a USB cover 116, and one or more air ducts 118 (e.g., “air flow holes”). The evaporator 100 is designed to be relatively small and portable, and is sized and shaped to fit comfortably in the hand or pocket of a user.

The housing 110 securely contains and protects many of the internal components of the evaporator 100 including but not limited to a heating element, power supply (e.g., battery), electronics, circuits, sensors, etc. The housing 110 may be composed of metal, ceramic, wood, plastic, and/or other materials that are sturdy and durable. In one embodiment, the housing 110 is composed of a material that has low thermal conductivity to minimize heat transfer from a heating element within the evaporator 100 to the outer surface of the housing 110 in order to prevent contact burns.

The suction cap 112 at first end 108 of the evaporator 100 contains an opening 122 within which resides a heating element (discussed in greater detail below) that supplies heat for evaporation of herbal products.

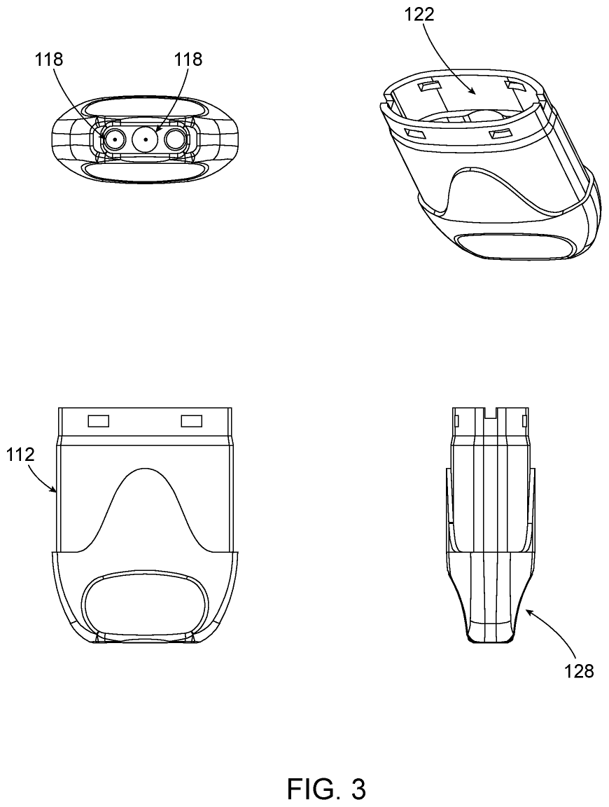

As shown in the illustrated example of FIG. 3, in one embodiment the suction cap 112 may have a curved shape similar to a bottom of an egg. That is, the suction cap 112 may have a semi-ovoid shape at the top and a v shaped window included in the design. This curved, semi-ovoid shape helps the suction cap 112 snugly fit within and couple to the evaporator's housing.

In one embodiment, the second end 114 of the evaporator 100 may include a USB connector (or any other type of connector not limited to USB) for charging a battery within the evaporator 100. The battery (not shown in FIG. 1, 2, 3, 4) supplies power to the heating element and other electronics. To protect the USB connector when it is not being used, the evaporator may include a cover 116 that removably couples to the second end 114 of the evaporator 100 thereby shielding the USB connector. In other embodiments, the second end may not include the USB connector or cover 116.

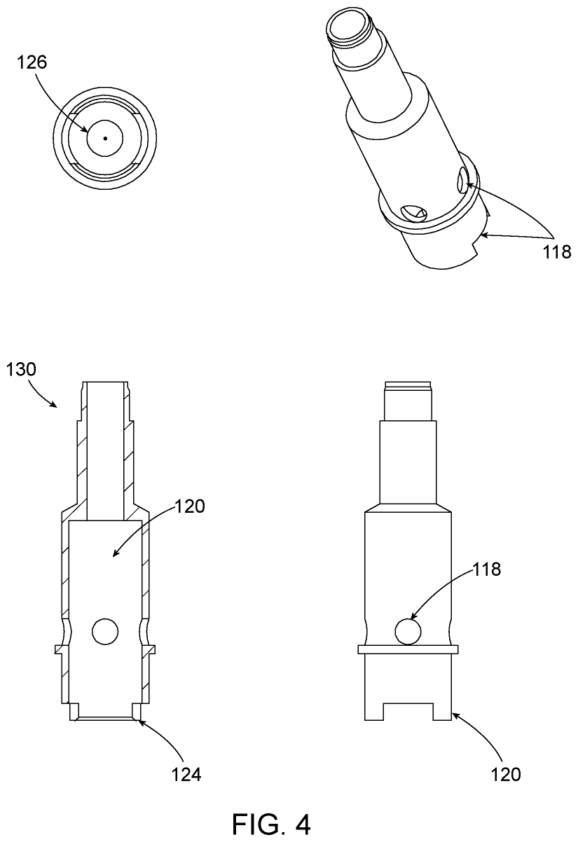

The air pressure differential in the chamber 122 of the suction cap 112 activates and deactivates (i.e., turns ON and OFF) the evaporator's heating element 120 shown in FIG. 4. The air ducts 118 in the suction cup chamber 122 and heating element housing 124 allow air from outside the evaporator 100 to flow into the evaporator 100. As explained in greater detail below, the air flowing into the evaporator 100 through the air ducts 118 is heated by the heating element before flowing out through the evaporator's first end 112.

FIG. 4 illustrates a cutaway, side view 130 of the evaporator heating element 120 according to one embodiment. In FIG. 3 the evaporator 100 may include a heating 120 and a heating element housing 124 that contains and secures the heating element 120. (The dashed lines of the heating element 120 indicate that it is contained within the heating element housing 124.) The heating element housing 124 may be made of porcelain, ceramic, and/or metal.

The heating element 120 may include a resistance wire heating element wrapped around in a coil. The resistance wire may be, for example, composed of nichrome (80% nickel and 20% chromium) or some other metal alloy such as but not limited to an iron-chromium-aluminum alloy. Electric current supplied by a battery (not shown in FIG. 4) internal to the evaporator 100 passes through the heating element 120 and because of the heating element's 120 resistance, the heating element 120 heats up. The heating element 120 may be configured to achieve and maintain temperatures ranging from 200 degrees to 2,000 degrees Fahrenheit. In one embodiment, the heating element 120 heats up to temperatures so that just outside the first end 126 of the heating element 120 (i.e., just past the wire mesh screen) the temperature ranges between 300 degrees and 500 degrees Fahrenheit. This way popular herbal products, such as marijuana, or CBD oil placed close to the first end 126 vaporize but do not burn.

In one embodiment, the heating element 120 is located a distance x millimeters (mm) away from the first end 126 where x may range between 1 mm and 20 mm. In some aspects, the heating element 120 may be positioned about 5 to 10 mm away from the first end 126 (i.e., 5 mm×10 mm). The distance away from the first end 126 of the heating element 120 is positioned may be dictated by the temperature desired to be achieved at or about the first end 126 where herbal products will be placed. For example, if higher temperatures are desired at the first end 126 the heating element 120 may be positioned closer to the first end 126 (i.e., x is minimized). By contrast, if lower temperatures are desired at the first end 126 the heating element 120 may be positioned further away from the first end 126 (i.e., x is maximized).

According to one embodiment, the air ducts 118 are positioned below the heating element 120 on the outer surface 124 of the housing as shown. That is, the heating element 120 is positioned in between the air ducts 118 and the first end 126. The air ducts 118 allow ambient air from outside the evaporator 100 to enter into the housing 124 just below the heating element 120. In one aspect, the ambient air enters into a chamber 122 within the suction cup 122 housing that contains the heating element housing 124 and heating element 120. This air is then heated as it flows past the heating element 120 and out through the first end's opening 126. The evaporator 100 may have any number (e.g., 1, 2, 3, 4, 5, 6, etc.) of air ducts 118 and the chamber 124 may be cylindrically shaped.

FIG. 4 illustrates a side and a slanted view of the evaporator 100 according to just one embodiment. FIG. 3 illustrates a top view, side view 128 and slanted view of the suction cup 112. Ambient air from outside the evaporator 100 enters into the housing 124 and/or the chamber 122 which may contain the housing 124 as shown. Once the air enters the chamber 122 and/or the housing 124, it flows through/past the heating element 120, which if turned ON, heats the air as it passes by. The heated air then exits out though the opening 118 of the first end 112 and may be used to vaporize herbal products that are positioned at or near the first end 126 of the heating element 120 of the evaporator 100.

There may be different mechanisms that cause the ambient air to enter into the chamber 124 and/or the housing 122 through the air ducts 118. In one embodiment, a low air pressure region relative to the ambient air is formed at the first end 126 caused by a user inhaling on a smoking apparatus coupled to the first end 126 of the evaporator 100. The low pressure region at the evaporator's first end 126 causes higher pressure ambient air outside the evaporator housing 124 to enter the chamber 122 and/or housing 124 through the air ducts 118.

FIG. 3 illustrates a cutaway 128, side view of the evaporator 100 suction cup according to one embodiment.

FIG. 4 illustrates a cross-sectional view 130 of the evaporator 100 atomizer. In the illustrated example, the heating element 120 may be shaped like a coil and may reside within the heating element housing 124.

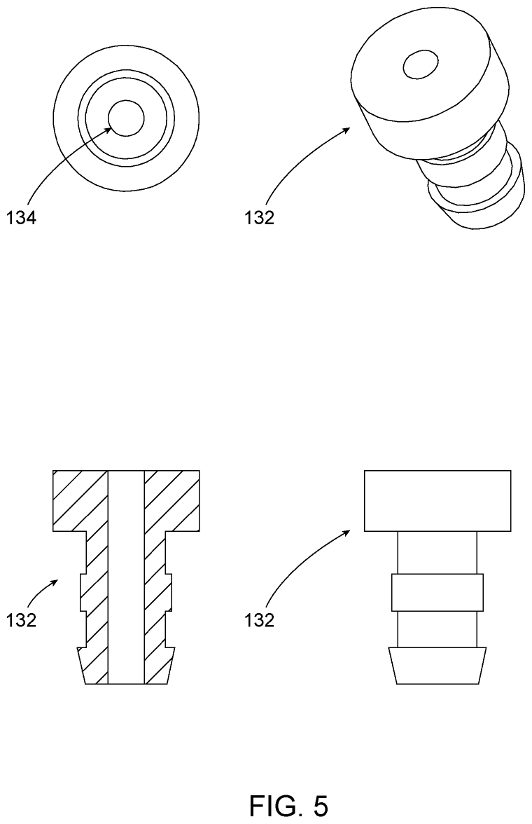

FIG. 5 illustrates a cross-sectional view 132 of the evaporator's 100 atomizer electrode.

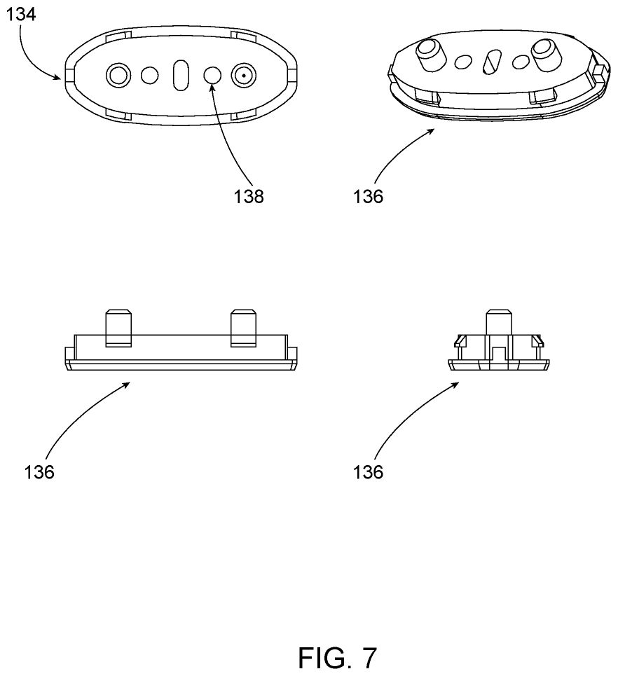

FIG. 7 illustrates a bottom view 134 of the second end 114 of the evaporator 100 with the cover 136 removed according to one embodiment.

The heated air drawn out from the first end 112 of the evaporator 100 is supplied by ambient air flowing into the evaporator's air ducts 118. As described above, this ambient air is heated as it flows past the heating element 120 (see FIGS. 3 and 4) within the evaporator 100.

As described above, ambient air enters the evaporator's air ducts 118 and is heated by the heating element 120 (see FIGS. 3 and 4) within the evaporator 100. The heated air then exits out of the evaporator's first end 126 and heats the herbal product so that it vaporizes. The resulting vapor then flows out from both top 118 and bottom openings 138 of the evaporator 100 and is eventually inhaled by the user.

The processing circuit 134 may execute instructions stored in the memory circuit. For example, the processing circuit 134 may control functionality of the heating element 120, various I/O interfaces, sensors, and the USB circuit 134. Activation of the heating element 120 may be regulated by the processing circuit 134, which may receive heating element temperature information from a temperature sensor coupled to the heating element 120. The I/O interfaces may include digital displays and/or LEDs that convey information pertaining to the temperature, battery life, and status of the evaporation apparatus 100. The I/O interfaces may also include one or more buttons or dials that turn the evaporation apparatus ON or OFF and/or set the heating element's temperature. The USB circuit allows an internal rechargeable battery to be charged through a USB connector.

One or more of the components, steps, features, and/or functions illustrated in FIGS. 1 through 7, may be rearranged and/or combined into a single component, step, feature or function or embodied in several components, steps, or functions. Additional elements, components, steps, and/or functions may also be added without departing from the invention.

The various features of the invention described herein can be implemented in different systems without departing from the invention. It should be noted that the foregoing aspects of the disclosure are merely examples and are not to be construed as limiting the invention. The description of the aspects of the present disclosure is intended to be illustrative, and not to limit the scope of the claims. As such, the present teachings can be readily applied to other types of apparatuses and many alternatives, modifications, and variations will be apparent to those skilled in the art.

What is claimed is:

1. A evaporating apparatus comprising:
an evaporating heating element; and
a housing encasing the evaporating heating element, the housing including a first end having an opening adapted to allow heated air provided by the evaporating heating element to exit out through the first end, the housing further including one or more air ducts on an outer surface of the housing, the one or more air ducts adapted to allow ambient air outside the housing to enter into the housing and flow past the evaporating heating element so as to be heated by the evaporating heating element, wherein the first end has a semi-ovoid shape that enables the first end to press against and substantially seal a substantially hemispherical-shaped bowl, and wherein the heated air enables evaporation of one or more herbal products contained in the substantially hemispherical-shaped bowl.

2. The evaporating apparatus according to claim 1, wherein the first end includes an elastic band that surrounds the opening and is adapted to press against and substantially seal the substantially hemispherical-shaped case.

3. The evaporating apparatus according to claim 1, wherein the evaporating heating element is positioned in between the one or more air ducts and the first end.

4. The evaporating apparatus according to claim 1, wherein the housing includes a second end opposite the first end, the second end including a Universal Serial Bus (USB) connector adapted to recharge a battery within the housing that provides power to the evaporating heating element.

5. The evaporating apparatus according to claim 4, wherein the second end includes a USB cover adapted to removably couple to the second end of the housing and cover the USB connector.

6. The evaporating apparatus according to claim 1, wherein the heating element is positioned between two (2) and twenty (20) millimeters away from the first end within the housing.

7. The evaporating apparatus according to claim 1, wherein the evaporating heating element is adapted to achieve a temperature ranging from 250 to 2,000 degrees Fahrenheit by heating and differential material composition.

8. The evaporating apparatus according to claim 7, wherein the evaporating heating element is adapted to achieve a temperature ranging from 330 to 500 degrees Fahrenheit by heating and differential material composition.

9. The evaporating apparatus according to claim 1, wherein the housing is substantially cylindrical-shaped.

10. The evaporating apparatus according to claim 1, further comprising:
a battery within the housing that supplies power to the evaporating heating element; and
a button coupled to the housing, the button adapted to activate the battery causing it to supply power to the evaporating heating element.

11. An apparatus comprising:
An evaporating heating element adapted to heat air; and
a housing encasing the evaporating heating element, the housing including a first end having an opening and at least one air flow hole, the evaporating heating element positioned between the opening and the air flow hole, and wherein a low air pressure region at the first end relative to ambient air outside of the air flow hole causes the ambient air to be drawn into the housing through the air flow hole and flow past the evaporating heating element to provide heated air out through the opening at the first end, wherein the first end has a semi-ovoid shape that enables the first end to press against an inner surface of a substantially hemispherical-shaped bowl to substantially seal the substantially hemispherical-shaped case, and wherein the heated air enables evaporation of one or more herbal products contained in the substantially hemispherical-shaped case.

12. The apparatus according to claim 11, wherein the first end includes an elastic band that surrounds the opening and is adapted to press against the inner surface of the substantially hemispherical-shaped bowl.

13. An apparatus comprising:
means for heating;
an input interface adapted to activate the means for heating; and
a housing at least partially enclosing the means for heating, the housing having a first end with a substantially semi-ovoid shape that enables the first end to press against an inner surface of a substantially hemispherical-shaped case to substantially seal the substantially hemispherical-shaped case, the first end having an opening adapted to allow heat from the means for heating to radiate out from the first end to evaporate one or more herbal products contained in the substantially hemispherical-shaped case.

14. The apparatus according to claim 15, wherein the first end includes an elastic band that surrounds the opening and is adapted to press against the inner surface of the substantially hemispherical-shaped case to create a substantially airtight seal.

15. The apparatus according to claim 14, further comprising:
a chamber within the housing; and
at least one air duct located on an outer surface of the housing that allows air to flow into the chamber through the air duct, and wherein the means for heating is positioned such that air flowing into the chamber from the air duct flows through the means for heating to be heated before it flows out through the opening.
